(19) United States
(12) Patent Application Publication
(10) Pub. No.: US 2008/0139846 A1
(43) Pub. Date: Jun. 12, 2008

(54) New Process 298

(75) Inventors: Lars Hansson, Karlskoga (SE); Martin Hedberg, Sodertalje (SE)

Correspondence Address: ASTRA ZENECA PHARMACEUTICALS LP;GLOBAL INTELLECTUAL PROPERTY, 1800 CONCORD PIKE, WILMINGTON, DE, 19850-5437, US

(73) Assignee: ASTRAZENECA AB, Sodertalje (SE)

(21) Appl. No.: 11/846,135

(22) Filed: Aug. 28, 2007

Related U.S. Application Data
(60) Provisional application No. 60/841,956, filed on Sep. 1, 2006.

(51) Int. Cl. C07C 233/57; C07C 209/00

(52) U.S. Cl. 564/123; 564/414

(57) ABSTRACT

A process for preparing a compound of formula I

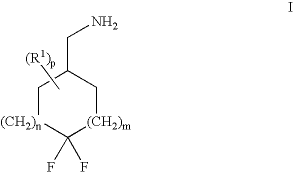

wherein R¹ is H, C₁₋₆alkyl, C₂₋₆alkenyl, C₁₋₆alkoxy, —OH, or amino; and n, m, and p are independently selected from 0, 1 and 2; which process comprises reacting a compound of formula II,

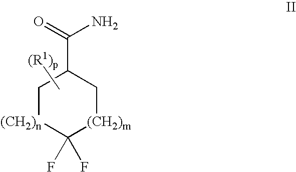

by combining it with a reducing agent in a suitable solvent into a reaction mixture.

### FIELD OF INVENTION

The present invention relates to a process of making a difluoro compound, and particularly to a process of making a difluoro compound containing an amino group.

### BACKGROUND

Difluoro compounds containing an amino group are useful intermediates in the synthesis of compounds having therapeutic effects. WO2004/108688 describes a method of making one of these difluoro compounds containing an amino group, [(4,4-difluorocyclohexyl)methyl]amine. However, an improved process of making these compounds is still desirable. It is in particular desirable to provide an improved process that contains a smaller number of steps and generates a higher overall yield.

### DESCRIPTION OF THE EMBODIMENTS

In one aspect, the present invention provides a process of making a compound of formula I

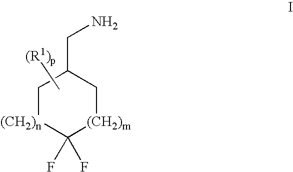

wherein R¹ is selected from hydrogen, C₁₋₆alkyl, C₂₋₆alkenyl, C₁₋₆alkoxy, —OH, and amino; and n, m, and p are independently selected from 0, 1 and 2; which process comprises re-acting a compound of formula II,

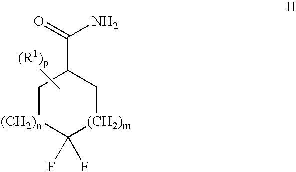

wherein R¹, n, m, and p are as defined in relation to formula I, with a reducing agent, by combining said compound of formula II with the reducing agent in a suitable solvent into a reaction mixture.

Throughout this patent application it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of trans-formation is limited only by inherent incompatibility of other functionalities carried by the molecule at that stage to the conditions or reagents employed in the transformation. Such inherent incompatibilities, and ways to circumvent them by carrying out appropriate trans-formations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4^{th} ed. McGraw Hill (1992) or, “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

As used here, the term “Cₘ₋ₙ” or “Cₘ₋ₙ group” used alone or as a prefix, refers to any group having m to n carbon atoms.

The term “hydrocarbon” used alone or as a suffix or prefix, refers to any structure comprising only carbon and hydrogen atoms up to 14 carbon atoms.

The term “hydrocarbon radical” or “hydrocarbyl” used alone or as a suffix or prefix, refers to any structure as a result of removing one or more hydrogens from a hydrocarbon.

The term “alkyl” used alone or as a suffix or prefix, refers to a saturated monovalent straight or branched chain hydrocarbon radical comprising 1 to about 12 carbon atoms. Illustrative examples of alkyls include, but are not limited to, C₁₋₆alkyl groups, such as methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, and hexyl, and longer alkyl groups, such as heptyl, and octyl. An alkyl can be unsubstituted or substituted with one or two suitable substituents.

The term “alkenyl” used alone or as suffix or prefix, refers to a monovalent straight or branched chain hydrocarbon radical having at least one carbon-carbon double bond and comprising at least 2 up to about 12 carbon atoms. The double bond of an alkenyl can be unconjugated or conjugated to another unsaturated group. Suitable alkenyl groups include, but are not limited to C₂₋₆alkenyl groups, such as vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl. An alkenyl can be unsubstituted or substituted with one or two suitable substituents.

The term “alkoxy” used alone or as a suffix or prefix, refers to radicals of the general formula —O—R, wherein R is selected from a hydrocarbon radical. Exemplary alkoxy includes methoxy, ethoxy, propoxy, isopropoxy, butoxy, t-butoxy, isobutoxy, cyclopropylmethoxy, allyloxy, and propargyloxy.

The term “amino” refers to —NH₂.

“RT” or “rt” means room temperature.

Unless otherwise noted, the term “catalytic amount,” as used herein, includes that amount of a component capable of either increasing (directly or indirectly) the yield of the product or increasing selectivity toward the product by the use of a substoichiometric amount of the this component.

In one embodiment, the reducing agent may be selected from sodium aluminium hydride, lithium aluminium hydride, diborane, sodium (dimethylamino) borohydride, borane-dimethylsulfide-complex, lithium triethylborohydride, lithium aminoborohydrides; sodium bis(2-methoxyethoxy)-aluminium hydride; sodium borohydride in combination with iodine or combined with other reagents as for instance described in “Advanced Organic Chemistry”, March, 4^{th} ed. McGraw Hill (1992); borane or its THF-complex, or a combination thereof.

In a particular embodiment the reducing agent is selected from lithium aluminium hydride, borane-dimethylsulfide-complex, borane or its THF-complex, sodium bis(2-methoxy-ethoxy)aluminium hydride, and diborane.

In one embodiment the compound of formula II and the reducing agent are reacted at a mole ratio between 1:5 and 1:1.5. In a particular embodiment the mole ratio between the compound of formula II and the reducing agent is between 1:3 and 1:2.

In one embodiment the organic solvent is selected from aromatic hydrocarbons, such as toluene;

aliphatic hydrocarbons, such as n-heptane;
ethers, such as diethyl ether, tetrahydrofuran (THF), 2-methyltetrahydrofuran, 1,4-dioxane or diethyleneglycol dimethyl ether
or a mixture of two or more of said solvents.

In one embodiment the total amount of solvents used is up to about 100 volume parts per weight of starting material.

In one embodiment of the inventive process is carried out at a temperature between −100° C. to +150° C.; in a particular embodiment of the inventive process is carried out between room temperature and +130° C.

In a certain embodiment the reducing agent is lithium aluminium hydride dissolved in a mixture of THF and toluene. For large-scale manufacture suitable concentrations may be in the range of about 4-20 weight percent. In a particular embodiment the THF/toluene solution contains 15 weight percent lithium aluminium hydride, calculated on the total weight of the solution.

In another embodiment the reducing agent is lithium aluminium hydride dissolved in diethyl ether.

In one embodiment, R¹ is selected from hydrogen and C₁₋₆alkyl.

In another embodiment, n, m, and p are each, respectively, 1 and R¹ is hydrogen.

According to one embodiment of the invention the compound of formula II is prepared by reacting a compound of formula III,

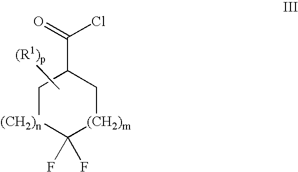

wherein R¹, n, m, and p are as defined in relation to formula I, with ammonia.

The ammonia may be used in gaseous form and/or in a suitable solvent. In one embodiment, the ammonia is present in a solvent selected from water;

aliphatic alcohols, such as methanol;
halogenated solvents, such as dichloromethane;
polar aprotic solvents, such as DMF or DMSO
and ethers, such as THF or 1,4-dioxane
or a mixture of two or more of said solvents.

In one embodiment the total amount of solvents used is up to 100 volume parts per weight of starting material.

In one embodiment the compound of formula II is prepared according to said process at a temperature between −100° C. to +130° C.; in a particular embodiment the temperature is between −20° C. and +100° C.

In one embodiment of the invention a solution of the compound of formula III is treated with an excess of at least 2.5 mole equivalents of aqueous ammonia.

In one embodiment, R¹ is selected from hydrogen and C₁₋₆alkyl.

In another embodiment, n, m, and p are each, respectively, 1 and R¹ is hydrogen.

The compound of formula III can be prepared by reacting a compound of formula IV,

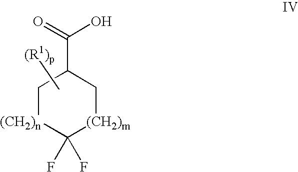

wherein R¹, n, m, and p are as defined in relation to formula I, with a chlorination agent.

The chlorination agent can be selected from thionyl chloride, oxalyl chloride, phosphorous pentachloride, phosphorous trichloride, phosphourus oxychloride, trichlorotriazine; triphenylphosphine in combination with carbon tetrachloride and/or trichloro acetonitrile.

The chlorination agent can be added to a suspension or a solution of a compound of formula IV in a suitable solvent containing a catalytic amount of N,N-dimethylformamide (DMF), N-methyl-2-pyrrolidinone (NMP), triphenylphosphine, one or more tertiary amine, such as triethylamine, tetramethyl urea, one or more quartenary ammonium salts, or N-formylmorpholine. The quartenary ammonium salts can be selected from tetraalkylammonium salts, such as disclosed in WO 96/36590.

The solvent can be selected from aromatic hydrocarbons, such as toluene; aliphatic hydro-carbons, such as n-heptane; ethers, such as THF, 2-methyltetrahydrofuran or diethyleneglycol dimethyl ether; chlorinated hydrocarbons, such as chlorobenzene or dichloromethane; and mixtures of two or more of said solvents.

R¹ can be hydrogen or C₁₋₆alkyl; n, m and p can be 1.

According to one embodiment of the invention the compound of formula II is prepared by first reacting a compound of formula IV,

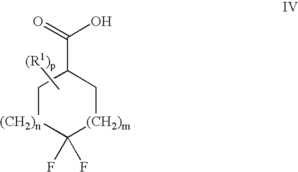

wherein R¹, n, m, and p are as defined in relation to formula I,
with a chlorination agent,
to produce a compound of formula III,

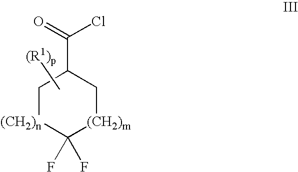

wherein R¹, n, m, and p are as defined in relation to formula I,
which is then, without being isolated, being brought to react with ammonia.

In one embodiment of the invention the chlorination agent is added to a suspension or solution of compound of formula IV in a suitable solvent containing a catalytic amount of DMF, NMP, triphenylphosphine, triethylamine, one or more tertiary amines, tetramethyl urea, quartenary ammonium salts, or formylmorpholine. In a particular embodiment said quartenary ammonium salts are selected from tetraalkylammonium salts as disclosed in WO 96/36590.

After removing excess reagent, sulfur dioxide, and hydrogen chloride, the cooled solution is treated with excess ammonia according to the procedures described above.

In one embodiment the chlorination agent is selected from thionyl chloride, oxalyl chloride, phosphorous pentachloride, phosphorous trichloride, phosgene, phosphourus oxychloride, trichlorotriazine, sulfuryl chloride; and triphenylphosphine; optionally with carbon tetrachloride or trichloro acetonitrile. In a particular embodiment the chlorination agent is thionyl chloride.

In one embodiment, R¹ is selected from hydrogen and C₁₋₆alkyl.

In another embodiment, n, m, and p are each, respectively, 1 and R¹ is hydrogen.

In another aspect the present invention relates to 4,4-difluorocyclohexane carboxylic acid amide.

In a further aspect the present invention relates to the use of 4,4-difluorocyclohexane carboxylic acid amide for the production of 4,4-difluoro-cyclohexanemethanamine.

### EXAMPLES

The invention will now be illustrated by the following non-limiting examples.

### Example 1

### Preparation of 4,4-difluorocyclohexane carboxylic acid chloride

4,4-Difluorocyclohexane carboxylic acid (4.45 kg, 27.1 mol), DMF (10 g, 0.1 mol) and toluene (10.5 l) were added to a glass-lined reactor previously rinsed with toluene. Thionyl chloride (3.30 kg, 27.7 mol) was then added during 22 minutes at 21° C. After one hour post reaction at 38° C. the reaction mixture was heated to 70° C. A sample was taken after one hour and forty-five minutes and submitted to gas chromatography analysis to confirm conversion of the starting material. The analysis revealed the presence of 2.8% residual 4,4-difluorocyclohexane carboxylic acid, detected as its methyl derivative.

### Example 2

### Preparation of 4,4-difluorocyclohexane carboxylic acid amide

The reaction mixture from Example 1 was allowed to stand over night at ambient temperature. Residual hydrogen chloride and sulfur dioxide were removed by distillation of toluene. Distillation was continued until the liquid temperature reached 115° C. After cooling, the acid chloride solution was transferred to and stored in a polyethylene container. The acid chloride solution was slowly added to a chilled aqueous solution containing 25 weight-%, based on the total solution, of ammonia (4.6 kg, 67.3 mol), during 67 minutes while maintaining the temperature below 40° C. After post reaction time of 30 min the product was filtered and washed with acetone. The product was dried as much as possible on a nutche filter over night. Then, the product was leached with water, re-filtered, and washed with acetone. Yield: 4.1 kg (92%). ¹H NMR (CD₃OD, TMS) δ 2.37-2.30 (m, 1H), 2.13-2.03 (m, 2H), 1.93-1.67 (m, 6H); ¹³C NMR (CD₃OD) δ 178.8, 122.5 (dd, J1=241 Hz, J2=239 Hz), 41.7, 32.6 (d, J=23.5 Hz), 32.2 (d, J=23.5 Hz), 25.6 (app d, J=10 Hz).

### Example 3

### Preparation of 4,4-difluoro-cyclohexanemethanamine

The wet 4,4-difluorocyclohexane carboxylic acid amide from Example 2 was charged to a clean vessel and dried under reduced pressure for 48 hours at a jacketed temperature of 100° C. Sampling and analysis showed less than 0.1% water. The vessel containing the dried 4,4-difluorocyclohexane carboxylic acid amide (4.1 kg, 25.1 mol) was charged with 19.5 l THF. The stirred suspension was sampled and analyzed for water content for safety reasons showing a 0.1% water content. A THF/toluene (2.4:1 w/w) solution containing 15 weight-%, based on the total solution, of lithium aluminum hydride solution (12.9 kg, 51.0 moles) was added to the suspension over a period of 100 minutes during which the liquid temperature ranged between 41 and 58° C. Evolution of hydrogen was produced during the first one third of the addition. After completed addition the vessel was closed and the temperature was raised to 69° C. The reaction was allowed to proceed for about 4 more hours. The reaction was then cooled below 0° C. and left over night. Then the reaction mixture was carefully quenched by the consecutive addition of water and diluted sodium hydroxide (0.3 kg, 7.3 mol) solution during three hours while the temperature was kept below 30° C. A second portion of water was added at 45-55° C. To maintain the temperature during the last addition external heating was necessary. During the addition of the sodium hydroxide solution the temperature increased to 50° C. The quenched reaction mixture was stirred for 10 minutes before filtration. The lithium and aluminum salts were washed with THF. The solvent was evaporated from the product solution under atmospheric pressure until the liquid temperature reached 115° C. The crude product solution was then divided into two batches and distilled at reduced pressure to give 4,4-difluoro-cyclohexanemethanamine as a colorless oil. Yield: 2.1 kg (56%). ¹H NMR (DMSO-d6) δ 2.51 (m, DMSO), 2.42 (d, J=6 Hz, 2H), 2.03-1.93 (m, 2H), 1.83-1.65 (m, 4H), 1.32-1.28 (m, 2H), 1.15-1.05 (m, 2H); ¹³C NMR (DMSO-d6) δ 124.6 (dd, J1=241 Hz, J2=239 Hz), 46.7 (two peaks with 2 Hz in between due to conformational flexibility), 38.4, 32.8 (d, J=22 Hz), 32.5 (d, J=22 Hz), 26.3 (app d, J=9 Hz). MS [M+H]⁺ 150.

1. A process for preparing a compound of formula I 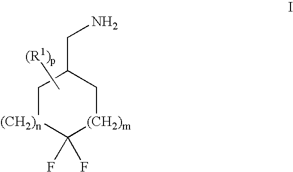
wherein R¹ is selected from hydrogen, C₁₋₆alkyl, C₂₋₆alkenyl, C₁₋₆alkoxy, —OH, and amino; and
n, m, and p are independently selected from 0, 1 and 2;
which process comprises reacting a compound of formula II, 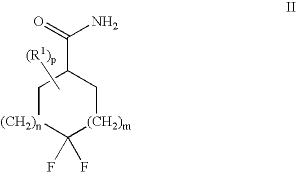
wherein R¹, n, m, and p are as defined in relation to formula I,
with a reducing agent, by combining said compound of formula II with the reducing agent in a suitable solvent into a reaction mixture.

2. A process according to claim 1, wherein R¹ is hydrogen or C₁₋₆alkyl.

3. A process according to claim 1, wherein n, m, and p are each respectively 1, and R¹ is hydrogen.

4. A process according to claim 1, wherein said reducing agent is sodium aluminium hydride, lithium aluminium hydride, diborane, sodium borohydride, optionally combined with iodine, sodium (dimethylamino) borohydride, borane, optionally in complex with THF or dimethylsulfide, lithium triethylborohydride, one or more lithium aminoborohydrides, one or more lithium trialkylamineborohydrides, lithium trimethoxy borohydride, or sodium bis(2-methoxyethoxy)aluminium hydride, or a combination thereof.

5. A process according to claim 1, wherein said reducing agent is lithium aluminium hydride; borane, optionally in complex with THF or dimethylsulfide; sodium bis(2-methoxyethoxy)aluminium hydride; or diborane.

6. A process according to claim 1, wherein said compound of formula II and said reducing agent are reacted at a mole ratio between 1:5 and 1:1.5.

7. A process according to claim 1, wherein said compound of formula II and said reducing agent are reacted at a mole ratio between 1:3 and 1:2.

8. A process according to claim 1 wherein the compound of formula II is prepared by reacting a compound of formula III, 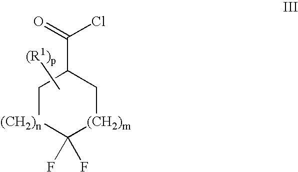
wherein R¹, n, m, and p are as defined in claim 1, with ammonia.

9. A process according to claim 8, wherein R¹ is hydrogen or C₁₋₆alkyl.

10. A process according to claim 8, wherein n, m, and p are each respectively 1, and R¹ is hydrogen.

11. A process according to claim 8, wherein the ammonia is provided in a solvent selected from water;
aliphatic alcohols, such as methanol;
halogenated solvents, such as dichloromethane;
polar aprotic solvents, such as DMF or DMSO;
and ethers, such as THF or 1,4-dioxane;
or a mixture of two or more of said solvents.

12. A process according to claim 8, wherein the process is carried out at a temperature between 0° C. and +100° C.

13. A process according to claim 8, wherein a solution of the compound of formula III is treated with an excess of at least 2.5 mole equivalents of aqueous ammonia.

14. A process according to claim 1 wherein the compound of formula II is prepared by first reacting a compound of formula IV, 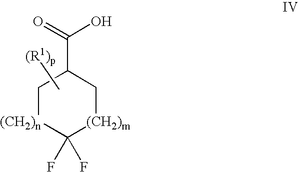
wherein R¹, n, m, and p as defined in claim 1,
with a chlorination agent,
to produce a compound of formula III, 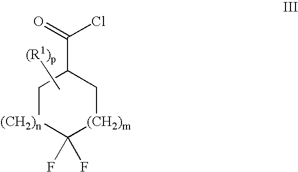
wherein R¹, n, m, and p as defined in claim 1,
which is then, without being isolated, being brought to react with ammonia.

15. A process according to claim 14, wherein R¹ is hydrogen or C₁₋₆alkyl.

16. A process according to claim 14, wherein n, m, and p are each respectively 1, and R¹ is hydrogen.

17. 4,4-Difluorocyclohexane carboxylic acid amide.

18. (canceled)
